(19) United States
(12) Patent Application Publication
(10) Pub. No.: US 2020/0367961 A1
(43) Pub. Date: Nov. 26, 2020

(54) ACNE TREATMENT SYSTEM AND METHODS

(71) Applicant: EXPLORAMED V LLC, Mountain View, CA (US)

(72) Inventors: Jonathan Podmore, San Carlos, CA (US); Joshua Makower, Los Altos Hills, CA (US); Earl Bright II, Sunnyvale, CA (US); Bryan Hartley, Redwood City, CA (US); John Hanley, Manhattan Beach, CA (US)

(21) Appl. No.: 16/992,225

(22) Filed: Aug. 13, 2020

Related U.S. Application Data
(63) Continuation of application No. PCT/US19/17984, filed on Feb. 14, 2019.
(60) Provisional application No. 62/671,329, filed on May 14, 2018.
(60) Provisional application No. 62/631,597, filed on Feb. 16, 2018.

(51) Int. Cl. A61B 18/14; A61B 18/20; A61B 17/3203; A61N 7/00

(52) U.S. Cl. CPC A61B 18/1477; A61B 18/203; A61B 2018/0047; A61N 7/00; A61B 17/3203

(57) ABSTRACT

Systems and methods for treating acne including an apparatus that applies or a method involving applying targeted energy to disrupt or destroy sebocyte progenitor cells within a target sebaceous gland. In one approach, specific sebocyte receptors are targeted to facilitate disrupting and/or destroying targeted sebocytes.

### FIELD OF THE DISCLOSURE

The present disclosure generally relates to systems and methods for treating acne.

### BACKGROUND OF THE DISCLOSURE

Acne vulgaris is a chronic skin condition of the pilosebaceous unit. It is characterized by the blockage of the hair follicle followed by inflammation and bacterial infection. The four pathogenic factors of acne are: i) hyperkeratinization of the cells lining the follicle; ii) increased sebum production; iii) Propionibacterium acnes “P. acnes”—a bacteria located on the skin; and iv) inflammation.

Acne is very common in young people as they enter their teenage years. During puberty, the sebaceous glands of the follicle grow reacting to the increased levels of hormones. This enlargement leads to increased sebum production. While there is some debate about the next step in the pathogenesis the result is blockage of the follicle with dead skin and sebum, called a comedone. P. acnes thrives in an anaerobic environment, which the clogged follicle has established. Additionally, P. acnes uses sebum as its primary source of energy. This combination leads to the papules, pustules and in more severe cases nodules and cysts.

The most effective therapies for treating acne are ones that directly impact sebum production. Accutane® (generic Isotretinoin) is considered a cure for acne and can result in a significant reduction in sebum production (over 80%) during the course of treatment. Isotretinoin is a systemic treatment and has significant side effects. Dryness of the skin, lips, eye, nose and mouth are most common and can be quite severe. Additionally, there are serious teratogenic risk and those on therapy must comply with a strict pregnancy prevention program. Anti-androgens (such as spironolactone) and oral contraceptives can help in clearing acne by regulating the hormones that drive the sebaceous glands to enlarge.

While other therapies for acne exist, they typically target the other factors of pathogenesis. As a result, they may help the mild to moderate sufferer but typically do not impact severe acne to the desired extent. Additionally, no other therapy is considered a cure for acne, merely a way to limit or manage the condition.

Working from the experience of Isotretinoin, a curative acne treatment should target sebum production. Other therapies such as Photodynamic Therapy (PDT) and laser treatments attempt to target sebum production by targeting the sebaceous glands but they do so in an indirect, poorly targeted manner. While this does have a clinical effect it is not to the extent of isotretinoin and comes at a cost of its own significant side effects such as erythema, swelling, skin peeling and pain during treatment.

The sebaceous gland works by the process of holocrine secretion whereby the basal, or progenitor cells, which line the sebaceous gland replicated and differentiate resulting in a sebocyte. A sebocyte will grow significantly in size as it accumulates lipids within. As new cells differentiate off the basal cells the lipid filling sebocytes progress to the opening of the sebaceous gland at which point the sebocytes rupture resulting in sebum. Sebum is the product of the ruptured cells and the lipids within. In order to develop a truly curative treatment for acne disruption of sebum production at the earliest level—the basal/progenitor cells are required. This disruption will reduce sebum production of the gland moving forward allowing for clearing of acne lesion and prevention of the pathophysical inputs for it to continue.

There is a continuing need for an effective and focused approach to treating acne. Moreover, there is a need for proactive treatment modalities that prevent future acne and which are easy and effective to use.

The present disclosure addresses these and other needs.

### SUMMARY OF THE DISCLOSURE

Briefly and in general terms, the present disclosure is directed towards acne treatment systems and methods. The system includes an apparatus that facilitates and a method involving disrupting up to even destroying sebocyte progenitor cells within a target sebaceous gland to reduce or eliminate sebum production. The apparatus can achieve disruption or destruction of the sebocyte cells via application of thermal, mechanical or biological means. In one approach, specific sebocyte receptors are targeted to facilitate cavitating and thermally ablating targeted sebocytes. In another approach, sebocytes are targeted with a cell-receptor targeting nanoparticle. A hand-held or robotic, powered acne scanning and sebocyte disrupting up to destroying device is provided to accomplish the desired acne treatment protocol.

In one particular aspect, the system includes a program that determines a pattern consistent with one or more aspects of a pilosebaceous unit (comprised of a hair follicle, sebaceous gland and arrector pili muscle) at an expected distance below the skin and/or an expected distance radially in association to one or more hair follicles, and determines a targeted location or locations of progenitor cells in relationship to the pattern. A targeted beam of energy is then delivered to the target progenitor cells to destroy or disrupt the cells, preferably without otherwise damaging the skin.

In another aspect, the system functions to create a predictive model based on histological evidence as to the highest probability of sebocyte progenitor cells location relative to a pattern of the hair follicle as a component of the pilosebaceous unit. A scanner is employed to determine the location of hair follicles and the scanner then can determine the highest probability of location of sebaceous gland and energy is applied to disrupt or destroy sebocyte progenitor cells.

The acne treatment system also includes in certain embodiments, separate scanning and mapping and ablation functionality. In one embodiment, high frequency ultrasound is utilized to create a map of hair follicles and/or sebaceous glands and then ablation energy is directed based upon the map.

Further, the acne treatment system is configured to track and identify signals specific to sebaceous glands. Treated areas are distinguished from untreated areas and subsequent treatments are directed towards untreated areas. The system is also configured to provide controlled doses of ablation to allow for a limited or selective ablation to thereby result in fewer ablations and patient impact over time. Moreover, ablation doses are lowered so that daily treatments are possible and to minimize the effects of ablative treatments.

In a further embodiment, means for assessing the significance of the particular sebaceous gland in terms of its degree of sebum production relative to other targets is further used to subselect the glands which are mostly responsible for excess sebum on the skin and/or most likely to be clogged or targets of bacterial infiltration. This sensing capability is further combined in the previously mentioned process and learning to lower the overall number of disruption sites and further reduce post-operative inflammation and damage.

In further embodiments, photoacoustic energy or thermal acoustics are employed at a specific wavelength to induce a wide band acoustic response which is received by a dedicated sensor or sensor array. Here, the lipids residing in the sebocytes are the basis for identifying the differentiation and progression of sebocytes towards sebum secretion. Where a laser imparts a photoacoustic effect, the laser additionally functions as an ablative source once a target sebaceous gland is identified.

In a particular method of treatment, topically applied contrast agents are used to identify sebaceous glands and to increase a photoacoustic response. Topically applied, prepared micro-spheres modified with receptors are also used to target specific receptors of sebocytes for facilitating precision ultrasonic cavitation in one or more treatment modalities. Such micro-spheres can additionally include gas and/or metallic particles that aid the ablation of the targeted progenitor cells.

Another system and method involve use of a needle or needles or an array of needles to penetrate skin to a fixed depth. Impedance measurements are taken of the tissue to locate target tissues such as that of a sebaceous gland. Monopolar and bipolar approaches to impedance measurement and tissue disruption is employed to treat or prevent acne. In one aspect, impedance measurements are mapped and analyzed to identify areas for treatment. A control unit commands a needle to deliver agents or energy to disrupt or ablate the sebaceous gland or specific portions thereof. In one or more embodiments, the system is configured to provide various approaches to reducing or controlling pain or tissue disruption. Lastly, focused laser or ultrasound can also be used in conjunction with the targeting means and methods stated above to produce the disruptive effect.

In another embodiment, the needle based system is used to penetrate the skin to a fixed depth and one or more needles are placed within or around the sebaceous gland using targeting means (imaging or alternatively with pattern recognition of the orientation of the pilosebaceous unit). The needles are then used to create mechanical disruption of the sebaceous gland and progenitor cells through air aspiration, pressurized air or water injection or through other mechanical means such as rapid rotational/cyclical movement of the needles with a specific pattern to preferentially target the gland and progenitor cells.

In yet another embodiment of a needle based system, the needles are used to penetrate the skin to a fixed depth, again targeting the sebaceous glands. Biologic material including drugs and sclerosing agents (such as sodium tetradecyl sulfate, ethanol, and hypertonic saline) is injected to disrupt (including destroy) the sebaceous gland and sebocyte progenitor cells. If a sclerosing agent such as sodium tetradecyl sulfate is injected into the sebaceous gland this will lead to a biological effect of destroying the epithelial cells lining the gland.

These and other features of the disclosure will become apparent to those persons skilled in the art upon reading the details of the systems and methods as more fully described below.

### BRIEF DESCRIPTION OF THE DRAWINGS

FIGS. 1A-D are flow charts, depicting functional aspects of various embodiments and approaches to an acne treatment system and method.

FIG. 2 is an enlarged view, depicting a hair follicle and surrounding adjacent tissue structure.

FIG. 3 is a side, partial cross-sectional view, depicting a treatment device and use of the treatment device on a skin surface.

FIGS. 4A-D are illustrative views of tissue, depicting one approach to identifying structures for acne treatment.

FIGS. 5A and B are illustrative views of tissue, depicting another approach to identifying structures for acne treatment.

FIGS. 6A-I are partial cross-sectional views, depicting employing particles to facilitate targeting structures for acne treatment.

FIGS. 7A-M are illustrative cross-sectional views of tissue, depicting steps in identifying and treating tissue facilitating acne treatment.

FIGS. 8A and B are illustrative cross-sectional views of tissue, depicting the identification of tissue structures for acne treatment.

FIG. 9 is alternative illustrative cross-sectional views of tissue, depicting the identification of tissue structures for acne treatment.

FIG. 10 is yet further illustrative cross-sectional views, depicting targeting tissue structures in an acne treatment.

FIG. 11A is a perspective view, depicting an automated acne treatment system.

FIG. 11B is rotated view, depicting use the automated acne treatment system of FIG. 11A.

FIG. 12 is an enlarged view, depicting an underside of a cartridge of the acne treatment system of FIG. 11A.

FIG. 13 is a side view, depicting another embodiment of an acne treatment system.

FIG. 14 is a perspective view, depicting a monopolar needle.

FIG. 15 is a perspective view, depicting a bipolar needle.

FIG. 16A is a perspective view, depicting a pair of monopolar needles.

FIG. 16B is a perspective view, depicting a pair of bipolar needles.

FIGS. 17A-B are cross-sectional and perspective views, depicting another embodiment of a treatment needle.

FIG. 18 is a cross-sectional view, depicting an acne treatment method involving taking an impedance measurement employing a monopolar approach.

FIG. 19 is a cross-section view, depicting an acne treatment method involving taking an impedance measurement employing a bipolar approach.

FIG. 20 is a schematic view, depicting employing an array of needles to take an impedance measurement.

FIG. 21 are images of mapping of impedance measurements.

FIG. 22 a cross-section view, depicting an acne treatment method involving generating

RF energy from a monopolar microneedle.

FIG. 23 is a cross-section view, depicting an alternate step of an acne treatment method involving generating RF energy from a bipolar microneedle.

FIG. 24 is a cross-sectional view, depicting disruption of tissue.

FIG. 25 is a cross-sectional view, depicting another approach to guided treatment.

FIG. 26 is a cross-sectional view, depicting employing a cooling cylinder in a treatment method.

FIG. 27 is a cross-sectional view, depicting inserting a treatment needle into tissue through the cooling cylinder of FIG. 26.

FIG. 28 is a perspective view, depicting an array of needles configured for targeted therapy delivery and cooling tissue.

FIG. 29 is a bottom view, depicting the array of needles of FIG. 28.

FIG. 30 is a cross-sectional view, depicting use of the array of needles of FIG. 28 in a treatment procedure.

FIG. 31 is a representative image, depicting use of tape identifying sebum expression.

FIG. 32 is a front view, depicting use of tape to identify the location of sebum production.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described. The present application claims priority to U.S. Ser. No. 62/631597, filed Feb. 16, 2018 and U.S. Ser. No. 62/671329, filed May 14, 2018, the entirety of which are incorporated by reference.

It must be noted that as used herein and in the appended claims, the singular forms “a”, “an”, and “the” include plural referents unless the context clearly dictates otherwise. Thus, for example, reference to “a sensor” includes a plurality of such sensors and reference to “the system” includes reference to one or more systems and equivalents thereof known to those skilled in the art, and so forth.

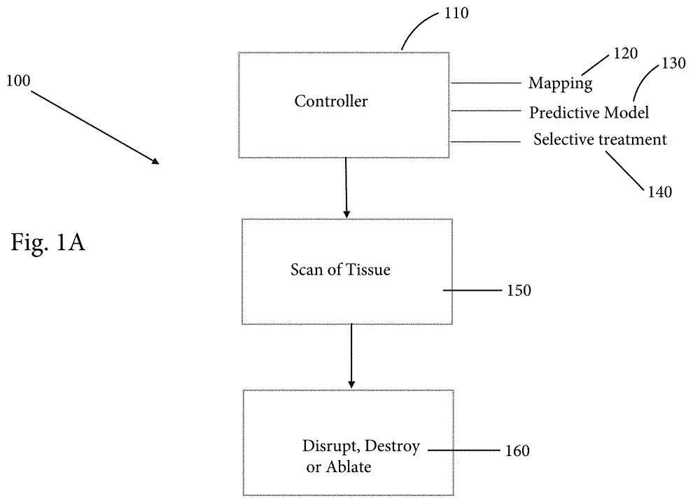

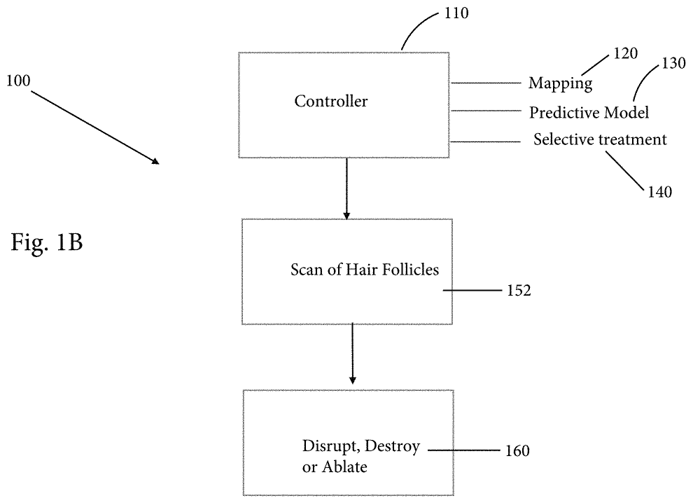

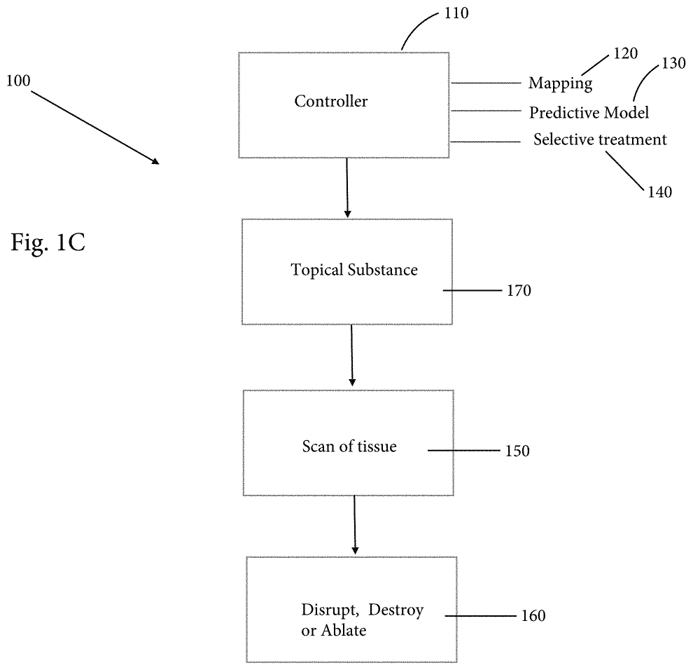

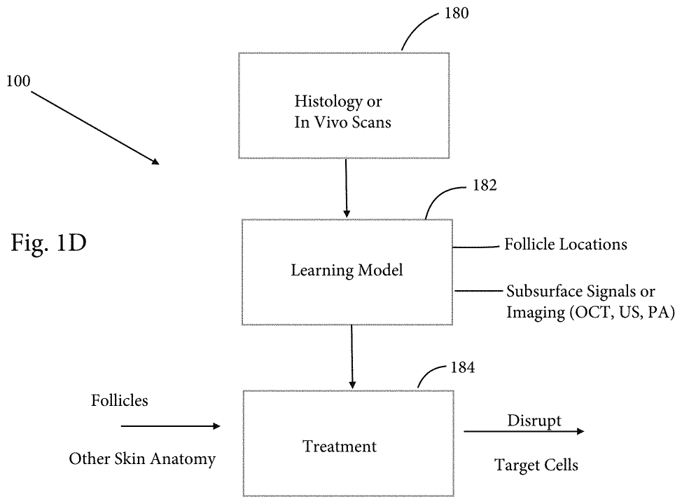

With reference to FIGS. 1A-D, there are shown steps or approaches in various embodiments or approaches to an acne treatment system and method 100 of the present disclosure. The goal of disruption of sebaceous glands is to return the patient to a state where acne is not a common occurrence. Acne sufferers typically produce more sebum then those without acne. While an individual may suffer from an overall general increased production from sebaceous glands, there is another possibility that selective glands provide the bulk of the overproduction. Where those glands and their attached follicles are identified, a route to a reduced amount of disruption and a treatment that is less impactful to the patient results. In addition to visual assessment of the hair and follicle, UV imaging is employed as P. Acnes, the bacteria related to acne, will fluoresce under UV light. Thus, the skin surface can be cleaned and then UV imaged after a set period resulting in an image (or targeting) of the follicles that display the largest amount of P. acnes fluoresce (which would correspond to sebum). These higher producing follicles can then be selectively targeted.

In one approach, as shown in FIG. 1A and described in more detail below, a controller 110 is provided to direct a desired treatment modality. In one aspect, the controller 110 is characterized by including a non-transitory computer-readable recording medium for storing and facilitating transmitting computer-readable instructions causing cooperating terminals or programs to execute communication processing with treatment devices and platforms. For example, the system 100 can be directed to map 120 a skin surface or structures below the skin surface of interest to identify acne or hair follicles or other skin structure or characteristics. The controller 110 can also direct the system 100 to assume one or more of a predictive model modality 130 or a targeted cell destruction 140 or other modality. The overall general treatment system or method 100 further includes scanning capabilities 150. This employs a signal-based approach to identify sebaceous glands or progenitor cells or additionally or alternatively, uses an image based approach for identifying sebaceous glands or progenitor cells. Also based upon the particular modality 130 directed by the controller 110, the system 100 accomplishes the desired disruption, destruction or ablation 160 to treat acne. It is to be noted that a complete scanning 150 of a treatment area can be conducted followed by disruption/destruction/ablation 160, or alternatively or additionally, scanning 150 and disruption/destruction/ablation 160 can be conducted as a combination or iterative treatment approach. In a related approach, as shown in FIG. 1B, a scan specifically of hair follicles 152 can be conducted, followed by or in combination with disruption, destruction or ablation 160. Here, the treatment works off a surface scan and follicular location, backed with either a predictive model or some other level of intelligent inference for targeting.

In yet another embodiment or approach to acne treatment systems or methods 100 (FIG. 1C), and as father described below, various topical substances 170 are applied to the treatment area prior to the scanning of tissue 150. Such substances include contrast agents that facilitate the identification of treatment areas, microspheres or carrier-based agents (e.g., liposomes) that aid in the selective treatment of specific areas or characteristics of skin by functioning as a signal enhancer. Thereafter, targeted scanning 150 and disruption, destruction or ablation 160 is employed to treat acne.

Moreover, as described in more detail below, acne treatment systems or methods 100 can also additionally or alternatively involve first conducting a histology or an in vivo scan 180 of a desired treatment area and further involves employing a learning model 182 responsive to or recognizing follicle locations or employing subsurface signals or imaging such as optical coherence tomography, ultrasound or photoacoustics. Subsequent treatment 184 is then based upon or responsive to the learning model 182 to thereby disrupt or otherwise treat target cells.

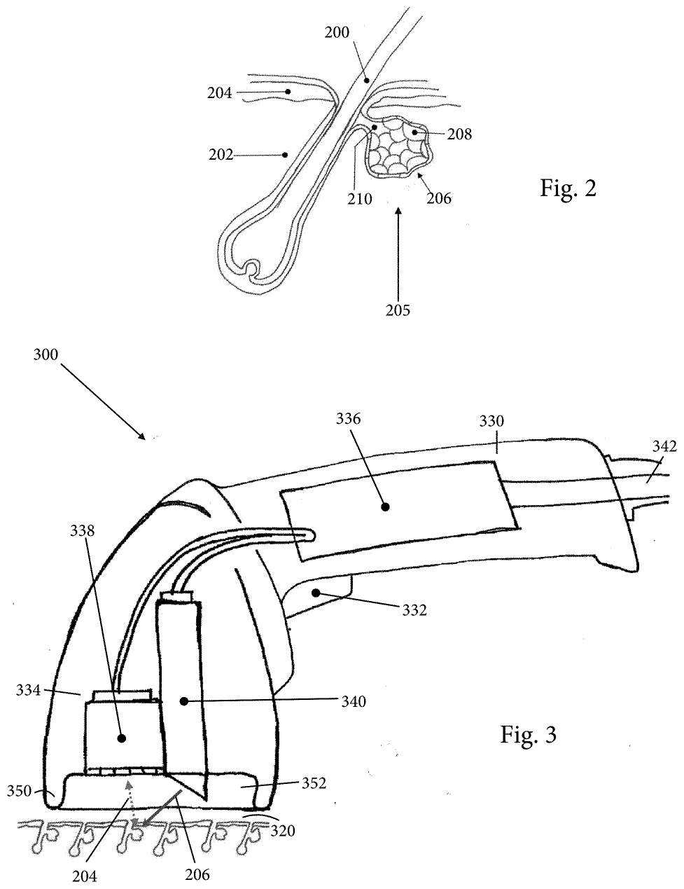

FIG. 2 depicts an enlarged view of a hair follicle 200 extending from dermis 202 through epidermis 204. Adjacent to the hair follicle 200 is the sebaceous gland 205 that defines a cavity lined with basal/progenitor cells 206. Within the cavity there are located sebocytes 208 and at the entry to the cavity is sebum 210.

An embodiment of a treatment device 300 is shown in FIG. 3. The treatment device 300 incorporates the controller 110 or communicates wirelessly or via a physical connection to controller 110, and can be self-powered and rechargeable. In use, the treatment device 300 is thus placed in proximity to or in contact with the area targeted for treatment 320. The treatment device 300 is passed over the target area 320 to accomplish various treatment modalities and objectives as directed by the controller 110. For convenience, the treatment device 300 includes a handle 330 that comfortably fits within a user's hand. An actuator or trigger 332 is configured on the handle 330 to control the operation of the device 300. Attached to the handle 330 is an enlarged head 334 for scanning and energy projection and detection. One or more buttons (not shown) are configured on the head 334 to select treatment modalities and to power the device on or off. A display (not shown) can also be provided to reflect the operational state of the device and to depict instructions to and with the controller 110. Within the handle is configured a PCB/Control board 336 that facilitates control of the operation of the device. In communication with the control board 336 and configured within the enlarged head 334 are a sensor/detector assembly 338 and a targeted therapy assembly 340. Extending proximally from the PCB/Control board 336 is a cable assembly 342 that can connect directly to a console (not shown). A frame 350 surrounds a window 352, the frame 350 being sized and shaped to comfortably engage tissue. The window 352 provides the conduit through which scanning energy and disruptive or destructive energy is projected to and from the target tissue 320.

In one particular approach to treatment, the treatment device 300 is configured to apply targeted energy to disrupt or destroy sebocyte progenitor cells within the sebaceous gland 205. Structure and electronics provided within the treatment device 300 generates one or more energy modalities, such as light or sound energy, for the purpose of delivering and receiving a signal to and from the targeted sebaceous gland 205. A program is provided and is capable of reading such signals and determining a pattern consistent with one or more aspects of the sebaceous gland 205 at an expected distance below the skin surface and an expected distance radially in association to one or more hair follicles 200. Machine learning or artificial intelligence is utilized to determine a predicted location or locations of the progenitor cells in relationship to the pattern and the follicles 402. Based on this analysis, a targeted beam of energy (light or sound based) is delivered to the progenitor cells to substantially destroy or disrupt them with minimal damage to the skin 300 above. FIG. 3 shows this scan/destroy concept for either a standard detection process or photo/thermal acoustic process. Sebaceous gland is sensed 404 by detector 338 and targeted therapy 406 is applied by laser 340, for example. In the photoacoustic embodiment, laser 340 illuminates tissue 406 and sebaceous gland 205 or sebum 210 return signal 404 to detector 338. With signal confirmed to be sebaceous gland or sebum, laser energy is increased for targeted tissue destruction.

In one or more embodiments, a machine learning or artificial intelligence component makes decisions regarding how many cells to treat and their distribution to thereby minimize side effects to the therapy and any resultant inflammation or swelling. The program controlling the system in certain approaches utilizes memory and pattern recognition to avoid re-treatment over areas previously treated.

The acne treatment system or method 100 alternatively or additionally includes or involves applying targeted energy to disrupt or destroy sebocyte progenitor cells within the sebaceous gland through creating a predictive model based on histological evidence as to where the highest probability of sebocyte progenitor cells are expected relative to any pattern of hair follicles. A scanner employing optical or other energy is used to determine the location of one or more hair follicles and then a predictive model is applied to determine where treatment energy is to be applied so as to have a high probability of disrupting or destroying sebocyte progenitor cells within the sebaceous glands.

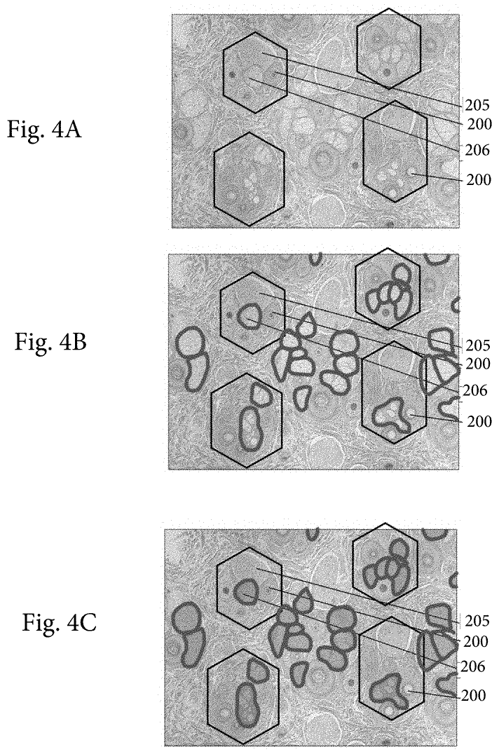

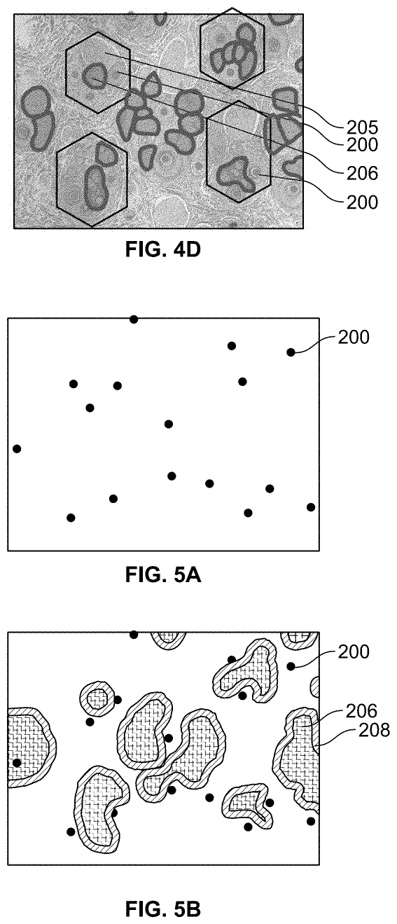

With reference to FIGS. 4A-D, there is shown one approach to treating acne employing machine learning and targeting. In a first step (FIG. 4A), a histological image at a depth corresponding to sebaceous glands 205 is taken. Next (FIG. 4B), employing machine learning, progenitor cells 206 of sebaceous glands 205 are identified and their locations are recognized and recorded. From the progenitor cell locations, sebaceous glands 205 are identified (FIG. 4C). Thereafter, as shown in FIG. 4D, using the location of follicles 200, relationships can be made for targeting of progenitor cells from follicular locations. Targeted cell destruction or disruption using one or more energy modalities then follows.

In a related approach, a fractional laser is employed, where a number of small lesions (called microthermal treatment zones (MZT)) are created on the surface of the skin in an organized (grid) or disorganized (random) pattern. The fractional laser can create random patterns and in some cases place lesions a set distance away from one another to minimize thermal load—but feedback of the skin and its structures are not feedback for treatment placement decisions. Next, an area or an area relationship around a follicle (or follicles) is determined with respect to the sebaceous gland to provide an option to greatly reduce the amount of ablation needed. For example, instead of 100% of the surface area, there would only be targeting of approximately 30%, or the region where sebaceous glands are believed to reside. Additionally, the system 100 is configured to track itself between treatments (on the same patient) so that it remembers follicular arrangements and potentially delivery a second treatment to areas missed in a first instance. Also, in the case of desiring a dose responsive treatment, the system 100 can be instructed to skip certain areas (possibly areas with active acne lesions) and return at a future treatment knowing the area that is untreated. Thus, fractional laser treatments avoid targeting the wrong locations and creating unneeded skin damage.

Turning to FIGS. 5A and B, in another approach, skin surface imaging can involve locating hair follicles 200 to identify sebocytes 208. As shown in FIG. 5A, a first step involves surface skin imaging identify follicles 200 (for example using the face that P. acnes in the follicle can fluoresce under UV light). Subsequently, the locations of the follicles 200 are used to define edges for progenitor cells 206 and thus, regions where sebocytes 208 are found.

In a further aspect, a topically applied contrast agent is used for selective photothermolysis (dyes such as methylene blue or indocyanine green) or ultrasound response (micro spheres or micro bubbles) when identifying sebaceous glands. Additionally, contrast agents can be modified to target specific receptors of the cells (sebocytes) of the sebaceous glands such that after an incubation period contrast is only located in the cells of the sebaceous glands.

Moreover, the present system and method 100 can additionally or alternatively include or involve using topically applied, prepared microspheres containing gas and modified to target specific receptors of sebocytes. For example, peptide hormone receptors including corticotrophin-releasing hormone, melanocortin, μ-opiate receptors, vasoactive intestinal peptide, cannabinoid receptors, histamine, insulin-like growth factor, growth hormone and CD44, nuclear receptors such as androgen receptors, progesterone receptors, estrogen receptors, retinoic acid receptors, vitamin D, perilipin 2, peroxisome proliferators-activated receptors, liver X receptors and vanilloid receptor, or other receptors including fibroblast growth factor receptors, epidermal growth factor, hepatocyte growth factor, CD14 and toll-like receptor can be targeted to enhance the acne treatment. In one aspect, the gas in the carrier is excited to rupture the carrier and release receptor clad gold or silver nanoparticles which will bind to sebocytes for targeted selective photothermolysis. Once so targeted, energy such as ultrasonic energy or IR laser heating is employed to cavitate or thermally destroy the targeted sebocytes or progenitor cells.

Additionally, in other approaches, topically applied, prepared liposome containing gas and metallic particles (gold or silver) and modified are utilized to target specific receptors of the sebocytes. The liposome is then ultrasonically ruptured to release the metallic particles adjacent to progenitor cells, and the progenitor cells are destroyed by infrared laser or other energy heating the metallic particles.

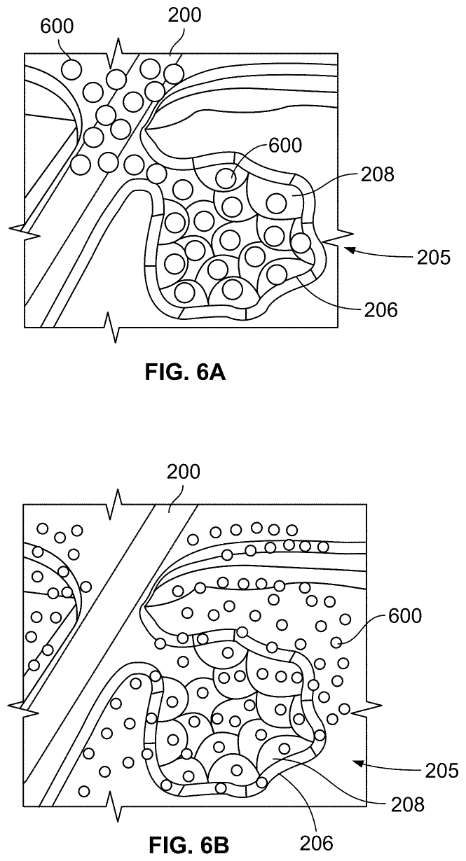

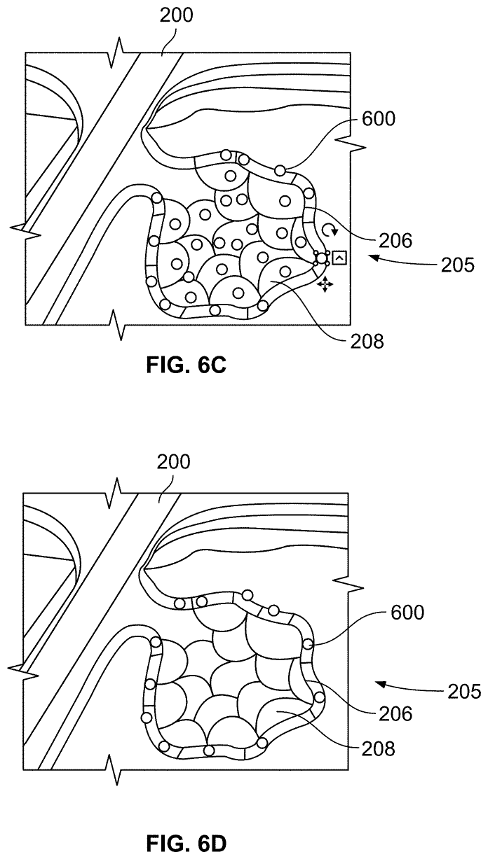

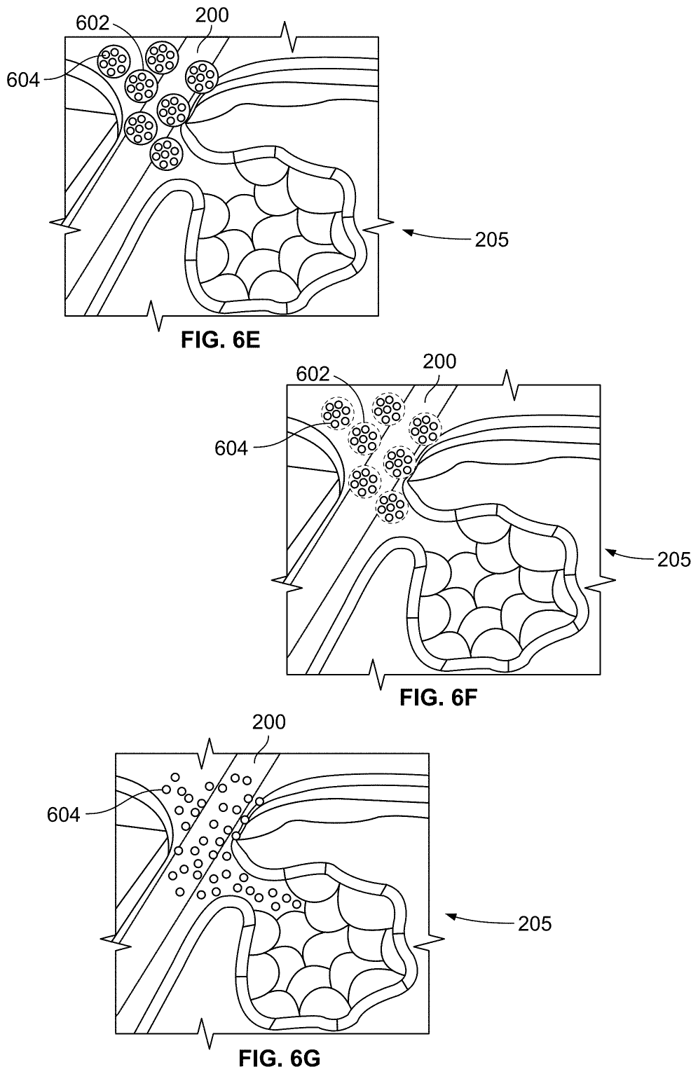

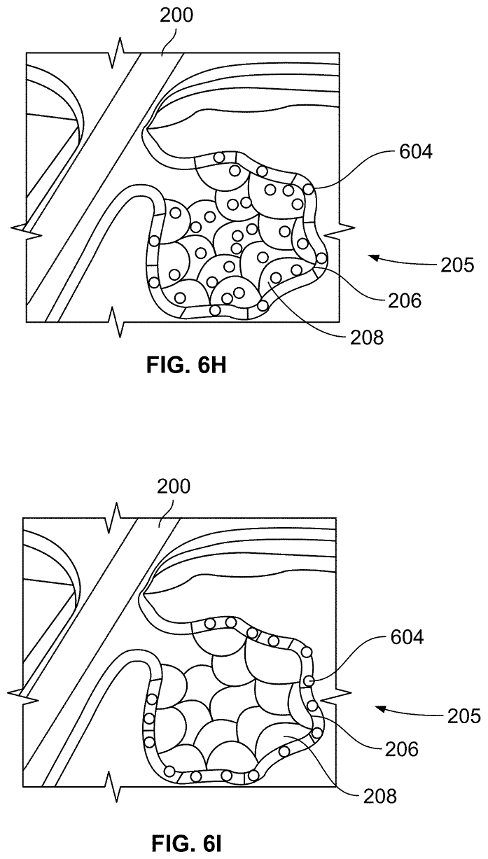

The acne system and method thus additionally or alternatively includes utilizing nanoparticles, for example metallic particles with receptors. The particles designed to penetrate and enter sebaceous glands to target sebocytes and progenitor cells so that IR laser heating, for example, creates local photothermally energized matter. Various approaches are depicted in FIGS. 6A-I. Such metallic particles with receptors are designed to be delivered transfollicularly with the objective of the metallic nanoparticles 600 targeting sebocytes 208 with specific receptors (FIG. 6A). A transdermal approach (FIG. 6B) can alternatively be taken with the result being to target sebocyte/basal cell specific receptors. The nanoparticles 600 can also be adapted to localize to specific sebocytes 208 due to receptors, where other cells in the area are free of particles (FIG. 6C) or to localize to progenitor cells 206 only (FIG. 6D). Acne treatment then proceeds with triggered release, such as with ultrasonic, infrared, protease, etc. triggering, of targeted therapy from a nanoparticle which penetrates the hair follicle. Targeted therapy could be a pharmacological agent or a targeted metallic particle.

In related approaches (See FIGS. 6E-I), a nanoparticle 602 constructed of smaller metallic nanoparticles 604 is employed so that it would be of a preferred size (for example, 600 nanometers) to enter the hair follicle, as shown in FIG. 6F. Once illuminated with a set level of energy (IR laser for example), the structure that holds them together will breakdown such that the smaller nanoparticles 604 are be released, thus resulting in better, localized penetration of the sebaceous gland and accumulation near the progenitor cells (FIG. 6G). Thus, as shown in FIG. 6H, the smaller nanoparticles 604 are adapted to target and accumulate within sebocytes. Also, the smaller nanoparticles 604 are adaptable to target progenitor cells 206. Here, in these approaches, receptors could aid in selecting target cells. In a particular aspect, the smaller, released particles can have surface receptors to specifically select targeted sebocyte or progenitor cells. These smaller, released nanoparticles are then targeted with the application of a higher level of IR laser energy to achieve the desired disruption or destruction.

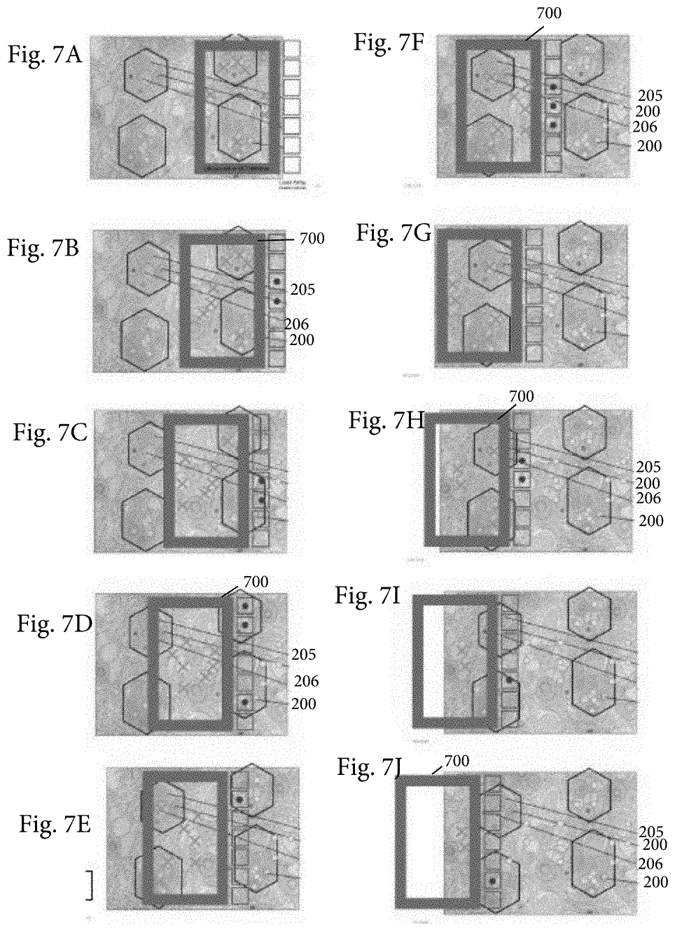

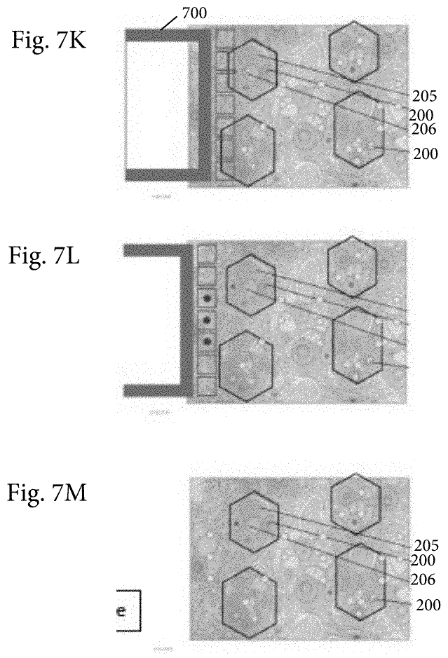

Separate scanning/mapping and targeted cell destruction approaches are also included in certain embodiments of the present system and method 100. In one approach to scanning tissue, as shown in FIGS. 7A-M, an initial sweep of tissue is performed to identify hair follicles 200, sebaceous glands 205, progenitor cells 206, and sebocytes or other anatomical structure. In accomplishing this scan, a window 700 of the scanner systematically slides across the target tissue while the system 100 notes and stores the location of the various tissue characteristics. Scanning and targeted cell destruction occurs in a single pass. The scan window 700 slides forward and the system 100 uses the returned scan data to establish targets for destruction (shown with X's)—locations of progenitor cells 206 or sebaceous glands 205. The scan and system 100 track the location of the targets such that it would be known when the targets pass into the target cell destruction zone 702 of the device. If a target area enters the target zone as the device is scanned across tissue, therapy (laser, ultrasound) is applied in a localized manner to treat or destroy the target tissue 704. This process repeats itself as the scanner is moved across the surface. Targets would be identified, and possibly tracked, when they present to the targeted therapy portion of the device therapy would be applied and the target tissue would be destroyed. In FIG. 7A-M, treated tissue areas are shown by the small dots. In one particular aspect, high frequency ultrasound (20 MHz to 120 MHz) is used to create a map of the sebaceous glands 204 on the face or other skin target area.

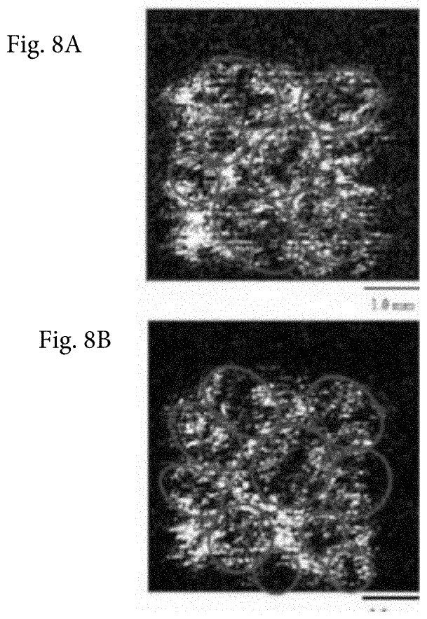

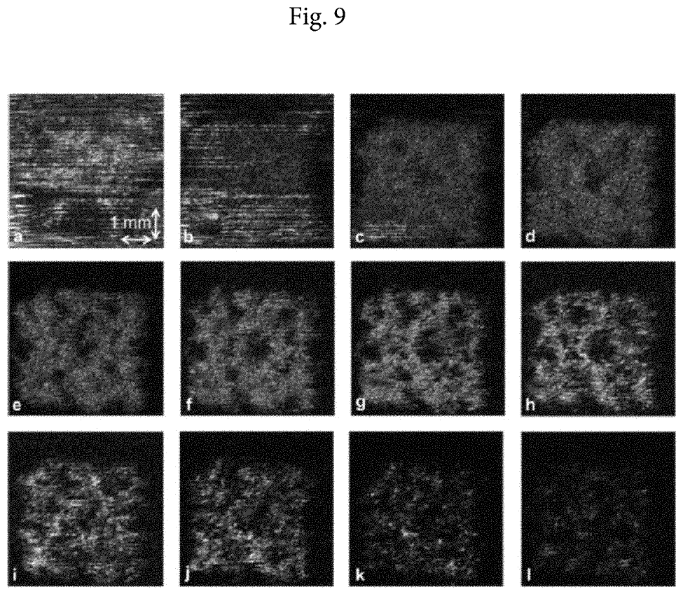

FIGS. 8A and B depict images achieved at a depth of about 800 to 900 microns, which is a characteristic depth of the sebaceous gland, using a 120 MHz ultrasound probe (single element transducer). The sebaceous gland is echolucent due to the lipid content so they appear dark/black in the figures. The edges of the sebaceous glands and potential locations for progenitor cells are shown highlighted. A set of images achieved using the 120 MHz single element transducer at a number of different depths is depicted in FIG. 9, where the dark/black areas appear due to the lipid content of the sebaceous gland and allow for estimation of the location both of the gland and the progenitor cells as well. In one embodiment, the treatment device 300 is a handheld structure with sensing capabilities to relate subsurface images to scanning orientation and location. The treatment device can also be embodied in a robotic arm so that locations are tracked as the arm algorithmically scans the target area.

With the map created, destructive energy is directed based on the recorded locations of the target sebaceous glands. The robotic arm itself or a robotic arm holding the handheld device 300 is controlled by the controller 110 or other controlling device to replicate and reproduce the mapped target locations with respect to the skin to direct energy. Also, the handheld device 300 can include an accelerometer or other position tracking mechanism which references the created sebaceous map for directed sebaceous gland or sebocyte destruction. Landmarks on the skin surface optically identified can additionally or alternatively be used to relate the sebaceous map to the treatment device 200 and identify treatment areas.

In another embodiment, a simplified ultrasound transducer with fixed focus at a specific depth (between about 0.5 and 1.5 mm) is incorporated in the treatment system 100. The intention here is to track and identify signals specific to sebaceous glands. With successful destruction of sebocytes and/or sebaceous glands, subsequent treatments would delineate treated areas from untreated tissue. Additionally, signals could be specific to untreated sebaceous glands such that treated glands respond similarly to background/non-target tissue. Additionally, or alternatively, the system 100 includes functionality where this transition of treated tissue would allow for a limited or selective dose of destructive energy delivery. That is, each treatment is 20% of treatable sebaceous glands—resulting in fewer subsequent treated targets and reduced patient impact with subsequent treatments. Moreover, the present system 100 can be configured so that doses can be lowered such that treatment could occur daily with a high or limited threshold for sebaceous targeting allowing for cumulative effects and minimal side effects of treatment. Notably, a signal-based approach would not require an imaging display system.

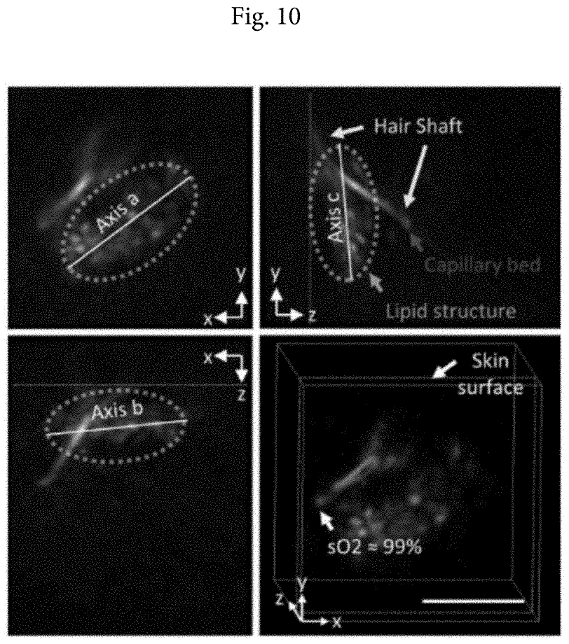

The acne treatment system and method additionally or alternatively includes use of photoacoustic or thermal acoustic energy. Such approaches facilitate identification of the sebaceous glands and/or progenitor cells. A series of photoacoustic images are shown in FIG. 10 of a pilosebaceous unit taken with two different wavelengths. A 1210 nm wavelength is shown in green and is the image of lipids within a sebaceous gland. The axis lines presented are an approximation of the limits of the gland and potential location of the progenitor cells of the sebaceous glands. Using a sebaceous gland selective wavelength (1210 nm or others) to induce a wide band (MHz) acoustic response which is received by a dedicated sensing transducer. The selective effect is a result of the lipid filled nature of sebocytes as they differentiate and progress towards sebum secretion. Lipids have a high number of C—H bonds which can be specifically stimulated.

Additionally, in the case of a laser imparting the photoacoustic effect the laser could additionally be the destructive source used once the sebaceous gland is identified. Thus, a method is provided where the skin is illuminated with selective wavelengths to the sebaceous glands, and received ultrasound energy at the transducer senses the photoacoustic response. In the case where the response is characteristic of a profile of a sebaceous gland, the laser energy is increased via a signal amplifier to the point of destructive injury to the sebocytes and/or sebaceous gland.

The acne treatment of the present disclosure also provide a needle or microneedle approach. As with each disclosed approach, machine learning or artificial intelligence component is employed to make decisions regarding how many cells to treat and their distribution to thereby minimize side effects to the therapy. The controller or program controlling the system in certain approaches utilizes memory and pattern recognition to avoid re-treatment over areas previously treated, involves conducting a histology or scans to facilitate directing treatment. The entire procedure (since visually guided) is monitored on a display where targeted follicles/glands are indicated and treatment durations and other variables are displayed while the treatment system is in use. Given that all of the locations of the treated glands are stored digitally and a unique fingerprint of the location of the follicles and treated regions are stored, the same data is usable as a reference for any subsequent treatments needed—either allowing for new glands to be treated and old ones not to be re-treated or to provide some means of efficacy/dosing assessment where the amount of sebum produced in a region can be demonstrated to be impacted correlated to previously treated regions and new regions needing treatment can be identified. All such data is stored and used for the machine learning algorithms. The system is further configured to be capable of tracking pitch/yaw and location of treatment structure in three dimensions relative to the treatment zone such that even the pace and locations of the treatment device is included in the data stored from the case. Moreover, this same visualization system can also be used to microscopically track progress of the lesions since it will be storing visual data and thus lesion resolution and size can be tracked under consistent and completely controlled lighting conditions within the enclosed space and a reconstructed map can be provided for physicians and core labs for analysis rather than the highly variable pictures at a distance of before/after at various time points.

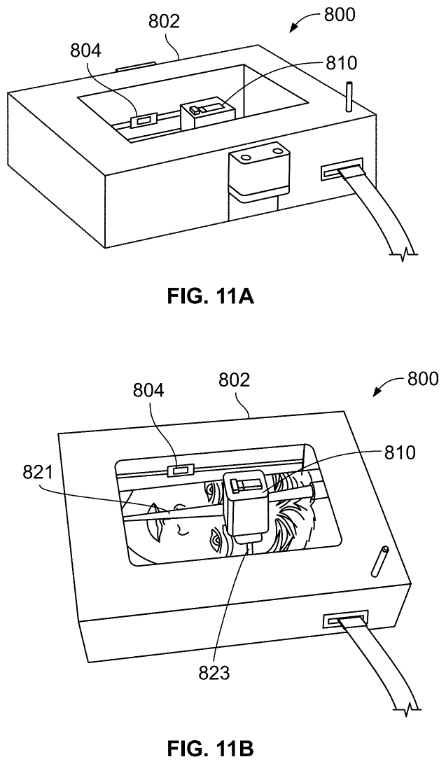

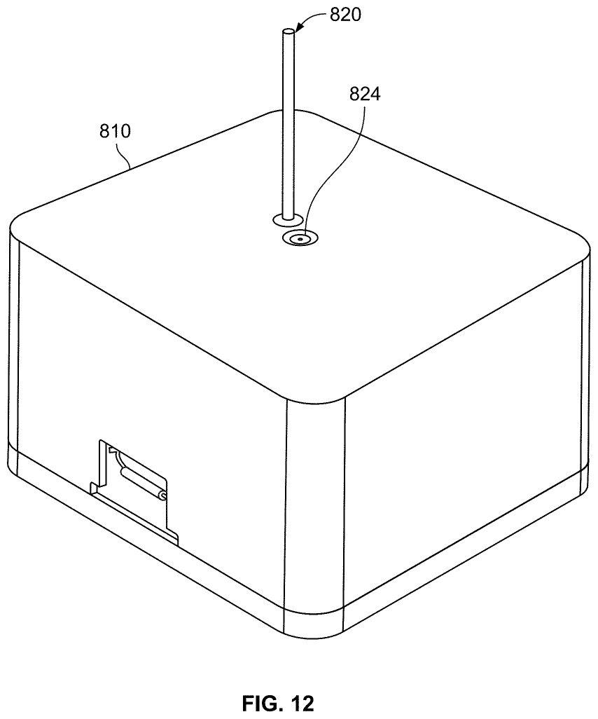

In one embodiment of the acne treatment system 100, as shown in FIGS. 11 and 12, there is provided an automated treatment delivery assembly 800. A patient with acne has anesthesia administered prior to treatment and is positioned relative to the automated treatment delivery assembly by an operator. The operator then starts the automated treatment system and the system performs the treatment. The automated treatment delivery assembly 800 can assume various shapes and sizes. In the embodiment shown in FIGS. 11 and 12, the assembly 800 defines a generally rectangular box or frame 802 including a window 804. The assembly 800 is large enough to span a face of an individual receiving treatments, but can assume for example, a smaller handheld configuration that is placed upon various portions of a patient's skin. The assembly 800 in a smaller version thus is configured to be used on any portion of a patient's body, or such that it only spans a portion of the area to be treated in order to administer to regions of the face such as the forehead, cheeks, nose, jawline, or the neck, shoulders or back. The assembly 800 or smaller version can further be attached to a stabilizing arm for assisting a user in placing the assembly 800 or robotic arm that is machine-controlled to provide directed treatment to such desired target areas. Further, the treatment mechanism can include a curved mechanism along which the treatment cartridge moves in order to accommodate curvature.

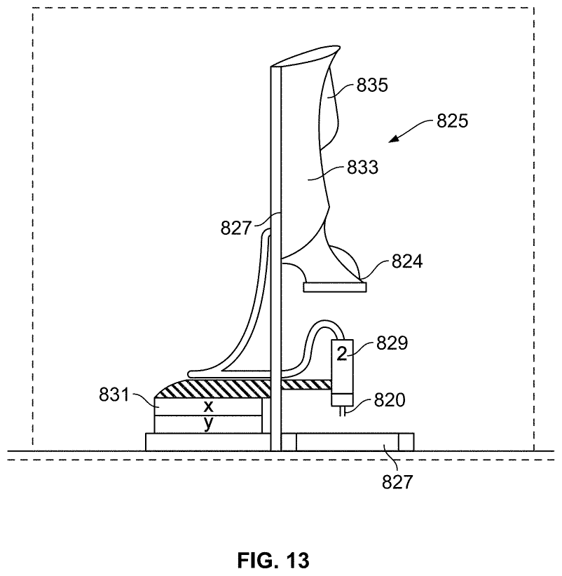

The automated treatment delivery assembly 800 includes a treatment cartridge 810 that is suspended from the frame 802 and configured to travel in a controlled fashion along two or three dimensions within the frame 802. A controller embodied in the frame 802 or remotely is provided to control the movement and speed of the cartridge 810 for treatment purposes. An underside of the cartridge (FIG. 13) is equipped with a microneedle 820 and a sensor or optical camera 824 that is employed to scan and communicate characteristics of the patient's skin to the controller. Translation screws 821 and 823 and stepper motors (not shown) move the cartridge 810 in the X and Y directions and actuator within the cartridge moves microneedle 820 in the Z direction. Rather than a single or plurality of microneedles, in alternative embodiments, the cartridge 810 is equipped with a high intensity focused ultrasound device, a surface RF device, a laser, larger needle or needles, or combinations thereof. In particular, an optical camera 824 is configured to identify hair follicles and distinguish hairs and an angle that they grow. As stated, the cartridge 824 can additionally include a second or additional microneedles 820. The cartridge 810 further includes a piston or other pivoting structure (not shown) that functions to extend and retract the microneedle 820 or project the microneedle 820 at various angles into the skin.

Referring to FIG. 13, there is shown another embodiment of a hand operated automated sebaceous or progenitor cell targeting microneedle system 825. In one particular aspect, the set-up and/or entire treatment region (expressed schematically with dashed lines) is contained within a semi or completely isolated cavity which contains one or more of the energy application apparatus, robotic and visualization means such that a consistent stream of extremely cold or super cooled air can flow against the skin during application of the energy to minimize damage to the skin and further numb the treatment region. There is a treatment window 827 provided such that a set area of the skin is treated. Here, the treatment area is 2.5 cm×2.5 cm but other embodiments could have different target treatment areas (1-2 cm and up). Where larger areas are treated, the system 825 is moved to new areas once the initial treatment is complete. The needle or needle array 820 is mounted on an arm 827 with the needle(s) 820 attached to a solenoid 829 for rapid movement in a z-axis. Alternatively, a hydraulic or pneumatic system can be provided for the z movement. The system 825 is characterized by embodying component parts that can be removed and replaced which allows for making it lighter and easier for the user to hold and manage. The needle array 820 is attached to an XY stage 831 to allow for movement around the treatment area. The stage 831 is motorized or can be manually moved. For target identification and tracking, a digital camera 824 is mounted directly above the treatment area with optics to visualize (including in the ultraviolet or infrared spectrum) the surface of the skin. While the system 825 is shown with a single camera element 824, in an alternative embodiment a dual camera arrangement is provided for stereoscopic viewing. Additionally, while the system shows a directly above location any other locations allowing for the camera 824 to fully visualize the treatment areas would be acceptable and in some cases may allow for better imaging during treatment preventing the treatment area from being blocked by the needle array 820 and arm 827. The camera area includes illumination for standardization of lighting. Additionally, there is provided a device cover that prevents any outside light from the treatment area for better light control—both for site imaging as well as if there are special lighting conditions needed (in the case of UV and fluorescence). An ergonomic handle 833 is further provided for holding and placing the device 825 by the user, and a treatment trigger 835 which activates the device and allows for the starting of treatment. For safety, the treatment stops based on the release of the trigger 835 as well as excessive movement tracked by the camera 824 or lower/loss of contact with the patient (pressure or skin sensors not shown).

In one aspect, there are provided a sensor-based microneedle or microneedles for both sensing sebaceous glands or progenitor cells and then delivering agents or energy to destroy or disrupt the sebaceous gland or progenitor cells. Either a monopolar or bipolar microneedle arrangement can be employed to generate radio frequency (RF) energy, for example.

In another embodiment, the needle based system is used to penetrate the skin to a fixed depth and one or more needles are placed within or around the sebaceous gland using previously mentioned targeting means (imaging or alternatively with pattern recognition of the orientation of the pilosebaceous unit). The needles are then used to create mechanical disruption of the sebaceous gland and progenitor cells through air aspiration, pressurized air or water injection or through other mechanical means such as rapid rotational/cyclical movement of the needles with a specific pattern to preferentially target the gland and progenitor cells.

In yet another approach, the needles are used to penetrate the skin to a fixed depth, again targeting the sebaceous glands. Biologic material including drugs and sclerosing agents (such as sodium tetradecyl sulfate, ethanol, and hypertonic saline) is injected to disrupt (including destroy) the sebaceous gland and sebocyte progenitor cells. If a sclerosing agent such as sodium tetradecyl sulfate is injected into the sebaceous gland this will lead to a biological effect of destroying the epithelial cells lining the gland.

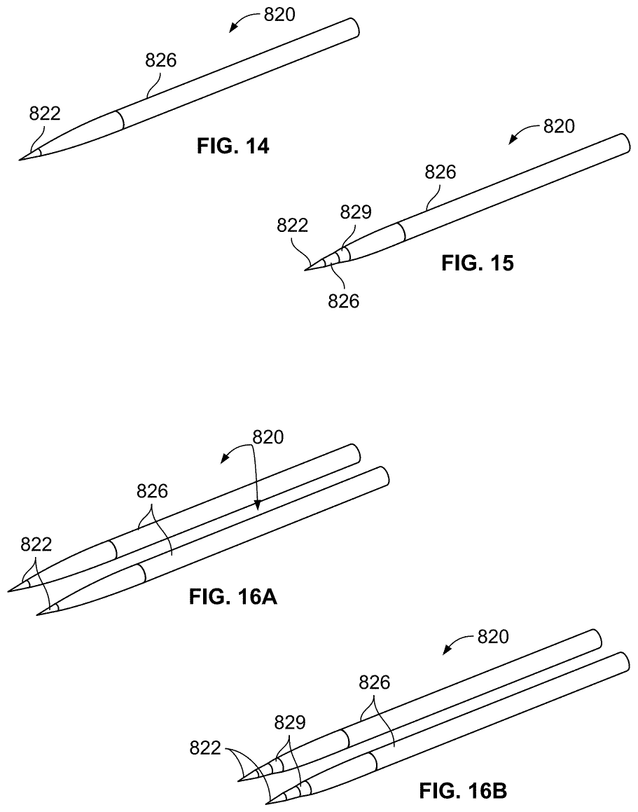

With reference to FIGS. 14-16B, the needles 820 are preferably approximately 200 microns in diameter, or in the range of approximately 100 to 300 microns, with a tapered point 822 over the distal about 1 to about 2 mm of length. As shown in FIGS. 14 and 16A, needles 820 can assume single and dual monopolar needle structure 824 where a dielectric layer 826 extends to the tip portion 822. FIGS. 15 and 16B depict single and dual bipolar needles 828 having two spaced dielectric layers 826 and an electrode 829 configured therebetween, and the tapered point 822 arranged distally thereof. Additionally, to isolate the treated area and generate precise impedance measurements the needles are insulated such that only a portion of the end between about 0.1 to about 1 mm) is electrically exposed. Insulations on the needles could be ETFE, PTFE, PFA, FEP, paralyene, polyimide, silicone or any other thin dielectric 826. Preferred needle array embodiments include a single insulated needle for monopolar, a bipolar single needle, two adjacent needles for bipolar (spacing between about 100 and 1500 microns), two monopolar needles, two bipolar needles, or four needles (mono/bipolar). As shown in FIGS. 14 and 16A, needles 820 can assume single and dual monopolar needle structure 824 where a dielectric layer 826 extends to the tip portion 822. FIGS. 15 and 16B depict single and dual bipolar needles 828 having two spaced dielectric layers 826 and an electrode 829 configured therebetween, and the tapered point 822 arranged distally thereof. In the case of the four or more needle array, such as four monopolar needles each attached to a ground or four bipolar needles, the concept would be to target the follicle and locate the array on the follicular opening. Once the needles are inserted to a prescribed depth there would be a needle in each quadrant or sub-section, and such needles are then energized as one, in series, or in a bipolar fashion.

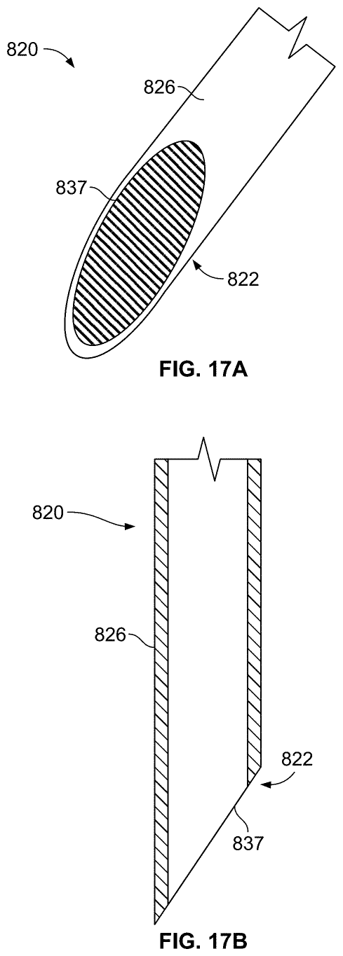

As shown in FIGS. 17A-B, to simplify the manufacture of needle electrodes, wire stock is used with dialectic 826 already applied before the setting or grinding of the needle tips 822. From the stock, the needle tip 822 is ground on a bevel or to a point 837. This would expose the core wire to act as the electrode while keeping the supplied dielectric 826 in place for electrical insulation and isolation. In one particular aspect, micro or nano fabrication manufacturing methods are employed to create treatment needles, and such dielectric material is sputtered onto an electrode substrate and thermally bonded so that it becomes resistant to peeling. The micro/nano fabrication approach also allows for a precision of sharpness of the needle tip down to a molecular level, thereby eliminating the need to rely on a conventional grinder.

A monopolar approach generally involves one energy generating element embodied in a microneedle, while a bipolar approach usually includes a pair of energy generating elements in its design, such as in a single microneedle (See FIG. 15). The monopolar element (See FIG. 14) is an active component when energized, and can require the application of another element functioning as a dispersive element elsewhere on the patient's body and operates to defocus or disperse the energy generated RF current, thereby preventing thermal injury to the underlying tissue. Like the monopolar element, the dispersive element is in direct communication with the energy generating unit and the same energy is transmitted across both the dispersive element and the active element. Bipolar microneedles generally are designed with two active elements; however, a bipolar microneedle can be designed such that one element is dispersive. The bipolar microneedle generates energy between the two active elements which eliminates the risk of current diversion and related adverse effects.

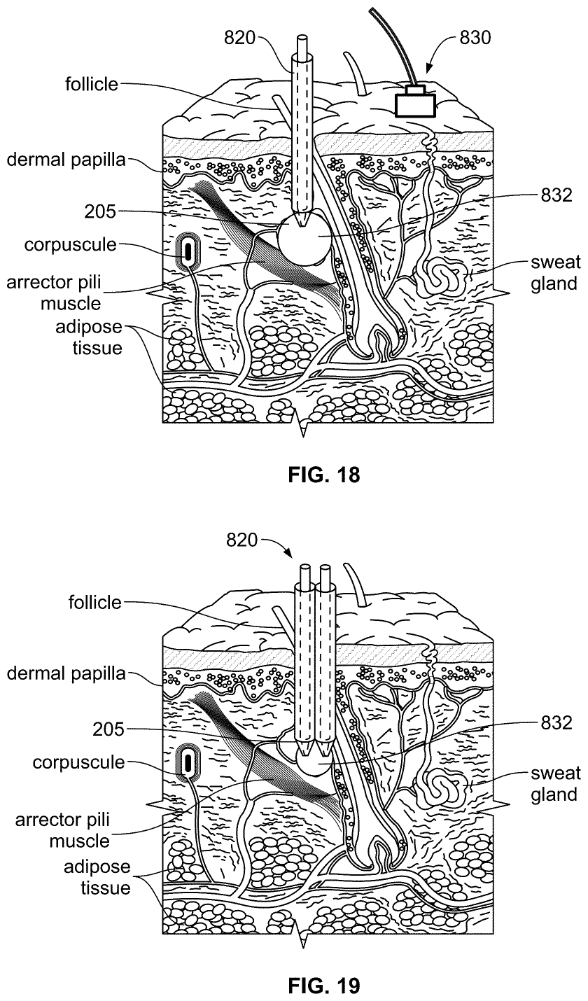

In a preferred embodiment, with reference to FIGS. 18 and 19, the skin is penetrated by the small gauge needle or needles 820 to a fixed depth, such as that corresponding to a location of a sebaceous gland or progenitor cells 205. The needle or needles 820 are configured to relay properties of the tissue near the tip back to the control unit. In the case of a property relayed that identifies a sebaceous gland or progenitor cells, the control unit commands the needle or needles 820 to deliver agents or energy to destroy or disrupt the sebaceous gland or progenitor cells. Here, the property can be impedance, a response to thermal input, conductance or capacitance from the surrounding tissue or to other needles.

One of the key features that differentiates the sebaceous gland from other tissue in the area of the hair follicle is that of the lipid nature of sebum being produced and accumulated in the sebaceous glands. Given the differences, impedance is used to determine the tissue that is near a measuring element such as a needle electrode—muscle is typically lower impedance (<2000 ohms) and lipid is typically higher impedance (>2000 ohms). Impedance can be measured using a number of parameters each of which has some impact on the resulting impedance measured. Based on benchtop work, impedances measured at 1 MHz (voltage peak to peak 50 mV to 2 V) produce consistence results. Frequency could vary from 1 kHz to 10 MHz. Impedance measurement can be monopolar or bipolar. In the case of a monopolar approach, the measurement is made between a needle and a ground pad. As shown in FIG. 18, the ground pad 830 is larger and attached to the patient. The monopolar approach typically involves measuring the impedance of tissue of an area 832 on the order of 4x the diameter of the needle. In the case of a bipolar approach, as shown in FIG. 19, the measurement area 832 is between the two needles and as a result can measure a smaller amount of tissue for impedance assessment. One preferred embodiment involves inserting an insulated needle pair 820 into the target tissue and bipolar impedance assessed. The resulting impedance reading indicates if the needles are placed in target tissue (SG), located in non-target tissue, or bridging a sebaceous gland or progenitor cells with one needle within the gland and one outside. Energy is then delivered based on an algorithm structured around the possible cases and potential impedance levels measured.

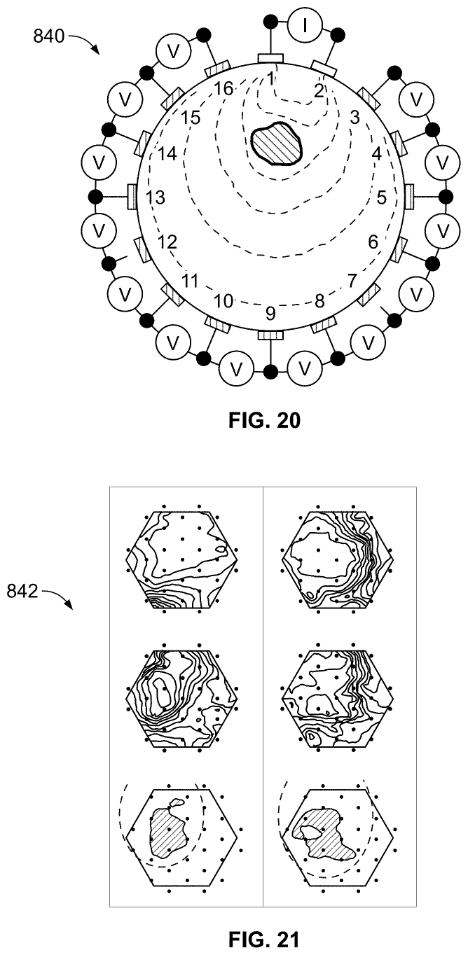

Thus, impedance is used to assess the tissue near the needle. With reference to FIGS. 20 and 21, an extension of this is the use of an array of needles 840 to create a map or maps of the area near the needles. More commonly referred to as electrical impedance tomography (EIT) an array of needles is used to create an electrical impedance “image” or images 842 of nearby tissue. The image is again based on the fact that impedance varies by tissue type. While a simple version of this would be to just measure impedance between all adjacent needles—there is also an embodiment in which two needles are excited for an impedance measurement between them while all the other needles are also monitored. Every adjacent needle pair is arranged in series and the resulting set of measurements can be analyzed to create a detailed map of the area. In a microneedle embodiment, an array of microneedles (for example, 7×7 or 8×8) could be inserted into the target tissue. In turn the impedance map is made of the nearby tissue. With the map in place targeting is determined based on the map potentially using mono-, bi- or multipolar energy delivery to precisely target the sebaceous gland highlighted on the impedance map.

In one particular approach, the microneedle 820 or pair or array is inserted into skin adjacent a hair follicle using optical targeting by using a camera to identify the location and angle of hairs. Alternatively, ultrasound or OCT or other surface imaging can be used to identify the location and angle of hairs. It has been discovered that a large portion of a sebaceous gland 205 is located below the area in which the hair is laying at an angle from the location where the hair follicle exits the skin (See FIGS. 18 and 19) and in some circumstances a small portion on the opposite side of the hair follicle. The pilosebaceous units consists of the hair, hair follicle, sebaceous gland and the arrector pili muscle (APM). The APM is the muscle that can move, or raise, the hair and cause “goose bumps”. Additionally, the APM is thought to play a role in advancing, or squeezing, sebum from the sebaceous gland out to the follicle and on to the surface of the skin. The source of this idea comes from the fact that the hair follicle and the APM make a triangle with the skin surface and typically within the acute corner between the follicle and APM is where the sebaceous gland lies. Some part of the gland might be opposite this but typically the larger portion of the sebaceous gland lie cradled in the APM. Additionally, this allows one to begin to estimate the location of the sebaceous gland. With this triangular arrangement the APM can be inferred to be “in plane” with the hair follicle and hair because the contraction of the APM will raise the hair upwards. Knowing it lies in this arrangement, identifying the angle of the hair as it leaves the follicle (as can be seen on the surface of the skin) the location of the sebaceous gland is approximated. Thus, targeting is based on visual assessment of the hair and/or follicle.

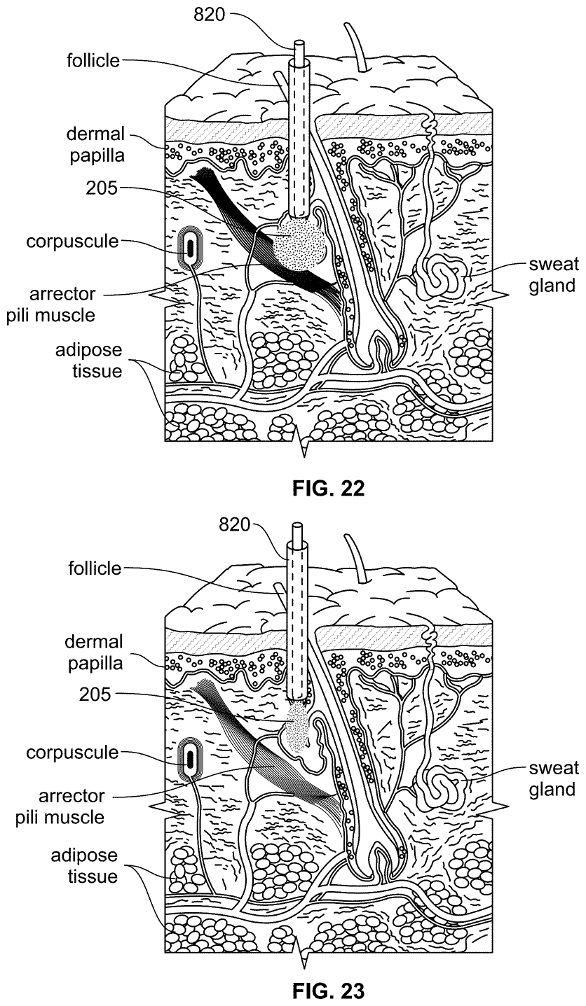

Accordingly, the major sebaceous gland lays below the acute angle between the hair and skin surface within an area defined by a “shadow” projected by the hair follicle under the skin. Ultrasound or laser-based imaging from the surface can be used to see the “shadow” or to identify the opening where the follicle exits the skin. Therefore, the microneedle 820 is inserted adjacent the follicle in skin residing below an acute angle defined by the follicle and the skin surface. The depth at which the microneedle 820 is inserted within the skin corresponds to the depth that a sebaceous gland 205 resides adjacent the hair follicle within the skin, for example from 800 to 1200 microns. After insertion at the target location (See FIGS. 22 and 23), the control unit commands the needle(s) 820 to deliver agents and/or energy to disrupt the sebocyte production. The control unit then retracts the needle(s), moves the needle(s) to the opposite side of the hair, inserts the needle(s) and commands the needle(s) to deliver agents and/or energy.

Where the impedance measured is on the order of 2000 ohms or greater, it is surmised that a sebaceous gland 205 is identified. Alternatively, the identification of a sebaceous gland 205 is achieved by looking for areas of higher relative impedance or resistance around a hair follicle. In a preferred approach, focused RF energy is then emitted from the microneedle 820 to thereby disrupt the located sebaceous gland 205. FIG. 22 depicts RF energy generated by the monopolar microneedle 820, whereas FIG. 23 depicts a more focused and controlled RF energy generated by a bipolar microneedle 820. The RF alternating energy heats target tissue by RF induced intracellular oscillation of ionized molecules that results in an elevation of intracellular temperature. When intracellular temperature reaches a target threshold, cell disruption occurs. It is to be recognized however that other forms of disruption energy can be employed as well such as laser energy or cryogenic energy (for example, in the form of a cooling fluid supply lumen extending distally within the microneedle, and a cooling fluid source connected to the supply lumen to direct cooling fluid flow into the needle lumen. When the flow is initiated, an outer surface of the needle engaging the target tissue may cool at a rate of about 25° C./sec to about 40° C./sec so as to promote intracellular ice formation and cell disruption). Disruption can also be thermal, via resistance heating of the needle, or can be chemical.

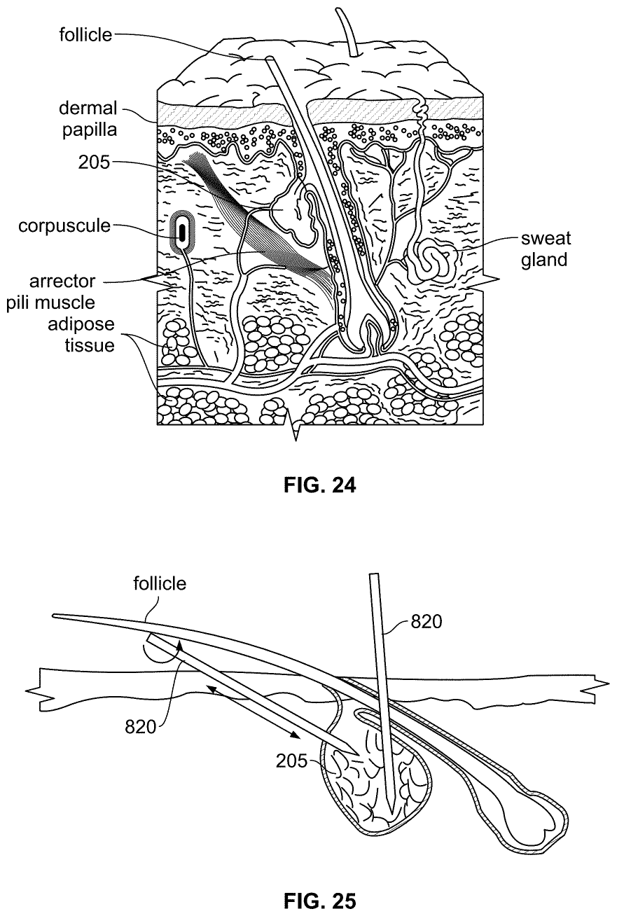

Once the sebaceous gland 205 is sufficiently disrupted (FIG. 24), the microneedle 820 is then repositioned on an opposite side of the hair follicle and an impedance measurement is taken once again. This step is taken because it has been recognized that a second sebaceous gland is often located on an opposite side of the hair follicle with respect to the first treated sebaceous gland 205. Where the system detects an impedance characteristic of another sebaceous gland, energy is again applied to disrupt the second sebaceous gland.

This process is repeated over an area of tissue that has been identified for treatment. Normal or tissue that has not exhibited inflammation is targeted and treated as well as tissue experiencing a break out of acne or gland hyper productivity. In one approach, a treatment has a goal of 80% reduction of sebaceous gland production for the targeted area employing an automated approach to treat and prevent acne. The use of an automated system has several advantages: more precise and controlled location of microneedle(s) to sebaceous glands; more efficient use of healthcare personnel and resources; ability to treat severe acne without toxic systemic drugs or long term use of antibiotics or hormone therapy; an approach involving one or two treatment needles significantly reduces the amount of energy employed and applied to tissue. Here, a targeted approach to treating sebaceous glands to the exclusion of surrounding tissue benefits the patient and results in reduced pain and recovery periods.

In a related approach (FIG. 25), treatment is based on the geometry of the pilosebaceous and placement of a dual needle array 820 or single needle. The needle electrodes can penetrate perpendicular to the skin surface or they can penetrate the skin surface at an angle and follow the trajectory of the follicle to reach the targeted region of the sebaceous gland. The needle electrodes 820 are placed in the sebaceous gland 205 using the anatomy of the follicle to aid in placement. The system provides both controlled vertical movement and rotational movement of the needles 820 to accomplished desired placement and directed treatment. The hair from the follicle is visualized and the dual needle array 820 is centered on the hair. This would place a needle electrode on either side of the hair. Then based on hair angle, follicle locations, and other anatomical characteristics the electrode penetrates the skin while still staying in the plane created by the hair. In one specific aspect, the needles 820 are placed such that a hair follicle is placed therebetween. The needles are then retracted or placed so that they are configured a set distance from a follicle opening based on the angle of the hair and an estimate of where the sebaceous gland starts along the follicle. When the needles penetrate the skin a detection method is employed to sense when needle tips are past the follicle and likely within the sebaceous glands. Sensing could be optical, electrical, or based on a characteristic of the needle such as strain, for example, in the case of the follicle pushing the needles outward.

In another approach, a needle or needles 820′ are additionally or alternatively used to create mechanical disruption of the sebaceous gland and progenitor cells through air aspiration, pressurized air or water injection or through other mechanical means such as rapid rotational/cyclical movement of the needles with a specific pattern to preferentially target the gland and progenitor cells. In yet another embodiment, biologic material including drugs and sclerosing agents (such as sodium tetradecyl sulfate, ethanol, and hypertonic saline) is injected through the needle(s) 820′ or by other means to disrupt (including destroy) the sebaceous gland and sebocyte progenitor cells.

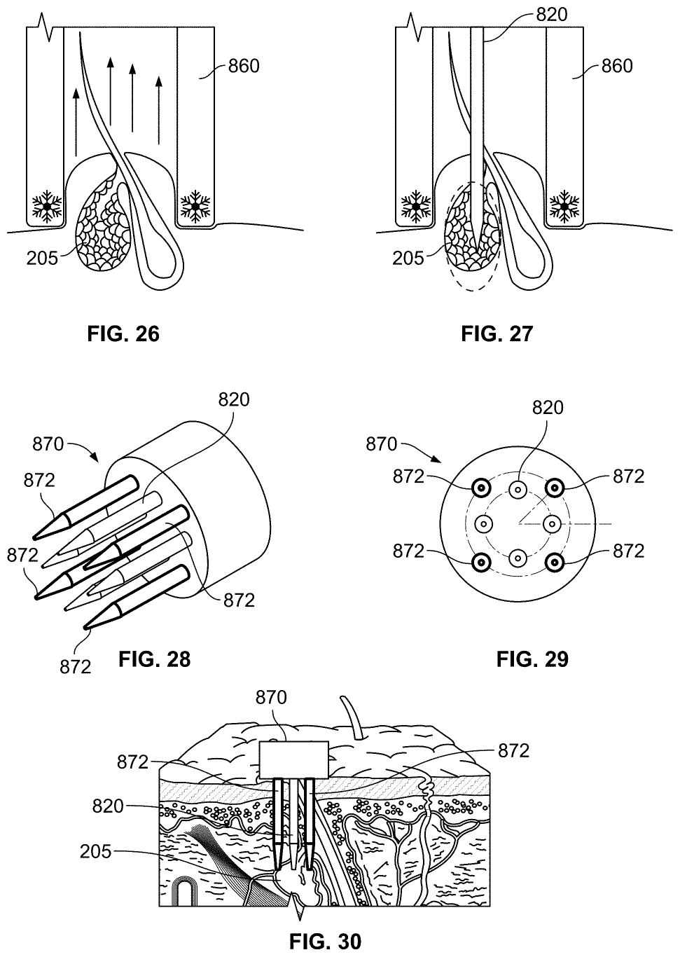

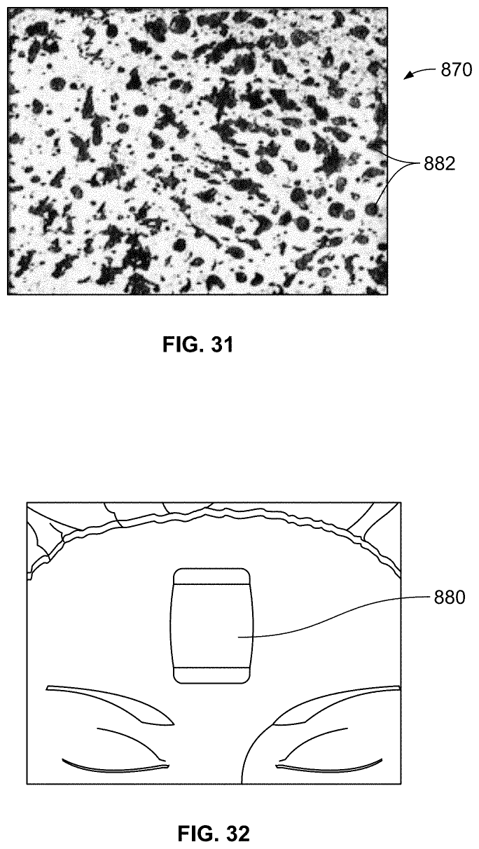

Additionally, in another aspect, cooling is employed along with controlled thermal disruption (See FIGS. 26 and 27). Cooling serves the dual purpose of both limiting the spread of any energy delivery/thermal disruption as well as acting to counter the pain signals generated by the energy delivery. Further, surface cooling could be used to prevent spread laterally and to the skin surface, thus protecting the epidermis. Here, the target tissue is drawn upward with a vacuum into a cooled cylinder 860. This functions to increase surface area for cooling. Additionally, this helps in tensioning the tissue for needle insertion and the negative pressure of the suction stimulates the pain receptors in the skin thus making them less sensitive to the needle penetration and the extreme heat or extreme cold used for cell disruption.

With reference to FIGS. 28-30, in yet a further aspect, where microneedles and microneedle arrays 870 are employed, some of the microneedles themselves are used to provide cooling to the tissues adjacent to the active needle electrode. In the case of an array 870, those needles that were not activated for disruption are cooled 872 to protect the surrounding tissue in the dermis between follicles and confine the extreme heat to the area of desired cell disruption. Additionally, in another approach, a simple array can have an encircling row of cooling electrodes to prevent the energy/thermal spread.

In yet a further aspect, there are shown in FIGS. 32 and 33 alternative approaches to identifying areas for treatment. As mentioned previously, there is interest in knowing which follicles are producing sebum (as it is not all) and possibly which are the excessive producers. Here, an adhesive tape 880 used to measure the amount sebum by absorbing the sebum into the tape 880 and making areas of the opaque tape transparent 882. It is to be noted that FIG. 32 depicts the tape 880 placed on a black background to show the locations 882 of the sebum producing follicles. The tape 880 can also be adapted such that alternative colors depict the presence of sebum. The tape 880 is used on a treatment site (FIG. 33) as an aid to guide which of the follicles are the largest contributor of sebum and allow for targeting of those follicles. In the more straightforward embodiment, the tape 880 is placed and therapy (laser or microneedle RF) is delivered through the tape 880. Additionally, the tape 880 is used for mapping purposes with the tape sebum areas mapped over an image of the target area highlighting the high producing follicular sites. Another related embodiment involves the application of a gel, foam, dye, or lotion that has an interaction with sebum such as turning or changing colors reflecting the detection or amount of detected sebum. In a related approach, sebum detection is exhibited as a fluorescence or as result of a chemical reaction for example, oligos designed to light up in the presence of sebum. Either visibly marking the follicles producing sebum or allowing for other imaging to highlight sebum producing follicles. In another embodiment, the tape is used to identify low sebum producing follicles that may be becoming blocked and beginning to form comedomes and acne lesions.

Accordingly, various approaches to acne treatment methods and apparatus are presented. The disclosed approaches are configured to provide an effective and focused approach to treating and preventing acne. The disclosed approaches can also be used to repair and reduce the appearance of acne scars in a targeted and automated manner. Further, the disclosed proactive treatment modalities are easy and effective.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

1.-52. (canceled)

53. An acne treatment system for treating target areas of skin including one or more hair follicles, comprising:
a treatment component that is configured to apply energy to target areas;
a sebaceous gland sensing component that identifies one or more of a location of sebaceous glands or progenitor cells thereof; and
a controller that automatically directs the treatment component based upon the location of sebaceous glands or progenitor cells.

54. The acne treatment system of claim 53, wherein the sebaceous gland sensing component employs impedance to identify target tissue.

55. The acne treatment system of claim 53, further comprising one or more of one or a plurality of monopolar or bipolar needles to measure impedance.

56. The acne treatment system of claim 53, wherein the treatment component includes a needle configured to apply RF energy to target areas.

57. The acne treatment system of claim 53, wherein a characteristic of impedance of sebaceous gland is employed to distinguish sebaceous gland tissue from surrounding tissue.

58. The acne treatment system of claim 53, wherein the system employs machine learning or artificial intelligence to determine a predicted location or locations of progenitor cells in relationship to hair follicles,

59. The acne treatment system of claim 58, wherein the machine learning or artificial intelligence makes decisions regarding the number of cells to treat and an associated distribution to minimize side effects to therapy.

60. The acne treatment system of claim 53, wherein the system includes a predictive model based on histological evidence as to probability of locations of sebocyte progenitor cells relative to a pattern of hair follicles.

61. The acne treatment system of claim 53, wherein the controller controls the system utilizing memory and pattern recognition to avoid retreatment over areas previously treated.

62. The acne treatment system of claim 53, wherein the system delineates treated areas from untreated tissue.

63. The acne treatment system of claim 53, further comprising one or more of a laser configured to impart photoacoustic energy for ablation once a sebaceous gland is identified or an ultrasound transducer with fixed focus at a depth and configured to track and identify signals specific to sebaceous glands.

64. The acne treatment system of claim 53, wherein the system is configured to specifically target progenitor cells rather than the sebaceous gland in total.

65. The acne treatment system of claim 53, further comprising an isolated treatment region, wherein a consistent stream of cold or super cooled air is projected against skin during treatment.

66. The acne treatment system of claim 53, wherein the system employs mechanical disruption of the sebaceous gland and progenitor cells through air aspiration, pressurized air or water injection or through other mechanical means such as rapid rotational/cyclical movement of the needles with a specific pattern to preferentially target the gland and progenitor cells.

67. The acne treatment system of claim 53, wherein the system injects biologic material including drugs and sclerosing agents (such as sodium tetradecyl sulfate, ethanol, and hypertonic saline) to disrupt (including destroy) the sebaceous gland and sebocyte progenitor cells.

68. The acne treatment system of claim 53, wherein the sebaceous gland sensing component employs UV energy to identify and target tissue.
